(19) United States
(12) Patent Application Publication
(10) Pub. No.: US 2005/0038301 A1
(43) Pub. Date: Feb. 17, 2005

(54) Chemical synthesis

(76) Inventors: Levan Kita Kavtaradze, Hamilton (NZ); Merilyn Manley-Harris, Hamilton (NZ)

Correspondence Address: FISH & RICHARDSON PC, 225 FRANKLIN ST, BOSTON, MA, 02110, US

(21) Appl. No.: 10/494,987

(22) PCT Filed: Nov. 8, 2002
(86) PCT No.: PCT/NZ02/00241

(30) Foreign Application Priority Data
Nov. 8, 2001 (NZ) 515366

(51) Int. Cl. C07C 29/10

(52) U.S. Cl. 568/867

(57) ABSTRACT

The present invention relates to a method of producing vicinal diols from a compound, the method characterised by the step of reacting the compound with a moderately strong acid in the presence one or more reagents capable of supplying hydroxyl groups wherein the moderately strong acid is a strongly reducing agent, but has a conjugate base that is a weak nucleophile. In preferred embodiments the moderately strong acid is hypophosphorous acid and the reagent(s) capable of supplying hydroxyl groups is 2-propanol in water, where 2-propanol is water soluable and organic. This method is particularly applicable to the production of vicinal diols of steroids, including lanosterol. Once vicinal diols of lanosterol diols are formed they are then capable of being further reacted to produce high purity lanosterol.

### TECHNICAL FIELD

This invention relates to a chemical synthesis.

More specifically, this invention relates to the synthesis of vicinal diols.

### BACKGROUND ART

The formation of vicinal diols from olefins is a widely researched topic due to their important chemical nature. Vicinal diols provide high value intermediates in organic chemistry, in particular, for the synthesis of biologically active compounds in optically pure form.

The traditional methods for producing vicinal diols from olefins often utilise highly toxic and highly expensive materials, or use materials that provide inferior yields or cause cleavage unless controlled.

Osmium tetroxide (OsO₄) and alkane potassium permanganate (KMnO₄) give syn addition of hydroxyl groups from the less-hindered side of the double bond. Osmium tetroxide adds hydroxyl groups rather slowly but almost quantitatively. The chief drawback to the use of OsO₄ is that it is expensive and highly toxic.

KMnO₄ is a strong oxidizing agent and thus may oxidize other functionalities in the substrate and unless conditions are carefully controlled can cause cleavage of the double bond, but under alkaline conditions treatment with KMnO₄ can produce vicinal diols. However, KMnO₄ has storage issues due to its strong oxidizing nature. It will support combustion of organics even in the absence of air and therefore cannot be stored in contact with organics. KMnO₄ is also very toxic to aquatic organisms and can cause long-term adverse effects in the aquatic environment.

Another syn addition to the double bond can be undertaken using thallium (I) acetate and thallium (I) benzoate. It should however be noted that thallium salts are poisonous.

It would be beneficial to have a method of producing vicinal diols that is not toxic, does not have storage, and therefore commercial production limitations, and is not as expensive as current practices.

It would also be beneficial to have a method of producing vicinal diols that has shorter reaction times than the standard OsO₄ reaction route.

The bulk of the discussion in this specification shall be directed towards the present invention in lanosterol and lanosterol derivative synthesis. Examples of other applications of the present invention are discussed later on in the specification.

Lanosterol is the core steroid from which others are derived by biological modification. It can be sourced from wool fat in sheep Merck Index, 10^{th} Edition, [1983]).

Lanosterol is included in a number of products, including cosmetics and de-inking materials. However, most of the interest in uses of stereochemically pure lanosterol derivatives seems to focus on two subjects: anti-fungal activity and steroid biosynthesis inhibition.

Fungal infections are a major clinical problem in infectious diseases, chemotherapy and immune-compromised individuals (e.g. AIDS sufferers). Current medications of choice are azole drugs, but resistance to these is now beginning to develop. The use of Polyene drugs for similar treatment have shown toxic side effects.

Both ergosterol and cholesterol the main sterols in fungi and mammals respectively are synthesized via lanosterol. C-4 and C-14 demethylations are common to both ergosterol and cholesterol biosynthesis, but C-24 methylation only occurs in fungi. Therefore recent works have identified in particular amino and thio derivatives of the side chain of lanosterol as potent anti-fungals due to their inhibition of the enzyme that brings about C-24 methylation.

Similar activity, which is due to inhibition of Δ^{(24,25)}-sterol methyl transferase, has also been demonstrated by 24,25-epiminolanosterol against Trypanosoma cruzi, the protozoan cause of Chagas Disease, a disease which gives rise to much human misery and economic loss in South America.

Commercially available lanosterol is a mixture of four closely related compounds, in which lanosterol (3β-hydroxy-8,23-lanostadiene) and dihydrolanosterol (3β-hydroxy-8-lanostene) predominate in the approximate ratio of 1:1.

Lanosterol is a highly desirable starting material for derivatisation to other steroids. Attempts have been made to separate lanosterol from dihydrolanosterol (and other impurities) by different methods. Unfortunately, common separation methods such as column chromatography or fractional crystallisation are almost impossible.

Earlier methods of isolating lanosterol from other sterols were based on the selective addition of bromine to the double bond in the side chain of lanosterol, isolation of the dibromo-derivative and debromination by sodium iodide in acetone or by zinc dust in acetic acid or benzene.

Low yields and impurity of the separated sterols have lead to the search for methods with improved yields.

The most successful results were obtained by Rodewald and Jagodzinski (Polish J Chem 1978, 52, 2473-2477). The reaction of acetylated commercial lanosterol with mercury acetate in aqueous tetrahydrofuran, followed by the in situ reduction of the mercurial intermediate with NaBH₄, provided a quantitative yield of 3β-acetoxy-5α-lanost-8-en-25-ol, which was separated from dihydrolanosterol by column chromatography, such as HPLC. Unfortunately, the use of HPLC is highly expensive which contributes significantly to the cost of the end product.

Also, mercury acetate is categorised as being poisonous and use of many mercury-based compounds is not preferred due to their detrimental environmental impact.

Alternatively, acetylated commercial lanosterol was selectively epoxidized at the 24,25-position, separated from dihydrolanosterol, and after reduction with LiAlH₄ and reacetylation, afforded 3β-acetoxy-5α-lanost-8-en-25-ol. Finally the 25-hydroxy derivatives were refluxed with 20% Ac₂O in acetic acid and 3β-acetoxylanosta-8,24-diene was obtained in 75% overall yield in relation to its content in commercial lanosterol.

LiAlH₄ is highly flammable and corrosive and reacts violently with water releasing flammable hydrogen gas. For example, the J. T. Baker Material Safety Data Sheet (MSDS) issues the following warnings about LiAlH₄:

“DANGER! CAUSES BURNS TO ANY AREA OF CONTACT. HARMFUL IF SWALLOWED OR INHALED. FLAMMABLE SOLID. WATER REACTIVE. MAY IGNITE IF HEATED OR CONTACTED WITH WATER OR ACIDS”.

Separate storage is recommended by the MSDS. It is obvious that LiAlH₄ would constitute a severe health and safety problem in large-scale operations.

More recently a solvomercuration-demercuration procedure has been provided as a general method for separation of unsaturated steroids as well as lanosterol from, wool grease. The steroid mixture containing unsaturated steroids was treated with organo-mercuric salts to give unsaturated steroid mercury compounds, which were converted to monoalkylmercury chlorides and then reduced to unsaturated steroids (JP 07258285 A2).

Again, mercury based compounds are not preferred due to their detrimental environmental impact.

Reports of successful separation of lanosterol from dihydrolanosterol are few, and none of them have solved the problem of the commercial production of pure lanosterol because the practical applicability of their approaches is limited by the hazardous and reactive nature of most mercury salts as well as reducing reagents.

As a result of this, pure lanosterol is prohibitively expensive.

At the time of writing, Sigma-Aldrich has available for sale lanosterol with a purity grade of 50-60% for 35.30USD for 25 g. Sigma-Aldrich also sells lanosterol with a purity grade of 97% for 46.60USD for 1 mg.

The discovery of a technically simple, environmentally acceptable separation technique for providing high purity lanosterol would have the advantage of providing a substantially pure source of product as a starting material for specific syntheses.

The discovery of a technically simple, environmentally acceptable separation technique for providing vicinal diols and in particular, those derived from lanosterol intermediates without recourse to HPLC would have the advantage of providing precursors to a number of other end products.

An example of an end product derived from a diol is a medical product, mephenesin, also known as Relaxil™, Renarcol™ or Tolserol™. This product is used as a skeletal muscle relaxant and is also used in the prevention of recurrent HIV-associated sinusitis. Formation of this product is by reaction with 3-chloro-1,2-propanediol and sodium o-cresolate.

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in New Zealand or in any other country.

It is an object of the present invention to address the foregoing problems or at least to provide the public with a useful choice.

Further aspects and advantages of the present invention will become apparent from the ensuing description that is given by way of example only.

### DISCLOSURE OF INVENTION

According to one aspect of the present invention there is provided a method of producing vicinal diols from a compound,

the method characterised by the step of

a) reacting the compound with a moderately strong acid in the presence one or more reagents capable of supplying hydroxyl groups
   wherein the moderately strong acid is a strongly reducing agent, but has a conjugate base that is a weak nucleophile.

The term ‘vicinal diol’ in accordance with the present invention means two hydroxyl groups severally attached to neighbouring carbons.

The compound may in some embodiments be a lanosterol intermediate such as a lanosterol derivative epoxide or hydroxyhalogenated lanosterol derivative or epimers thereof, although these are listed by way of example only and should not be seen to be limiting.

Other compounds may include 1,2-epoxycyclohexane, 2-halo-cyclohexanol, 2-bromo-1,2-diphenylethanol or epimers thereof, but these are listed by way of example only and should not be seen to be limiting also.

The term ‘moderately strong acid’ in accordance with the present invention should be understood to mean an acid with a pKa of less than or equal to 2.0.

The reagent may be water, or a number of liquids or combination thereof that are capable of providing hydroxyl groups.

Preferably, the reagent is water soluble and organic. The reagent should not be a competing nucleophile, which could give rise to side reactions.

In preferred embodiments the water-soluble organic reagent is 2-propanol. Other organic reagents such as methanol or ethanol might be used but there is a risk of alkylation, rather than hydroxylation, with primary alcohols due to methanol and ethanol acting as competing nucleophiles.

The term ‘weak nucleophile’ in accordance with the present invention should be understood to mean that the reagent in question, for example being either the conjugate base of hypophosphorous acid or the reagent containing the hydroxyl, does not attack the target carbon more readily than the incoming water molecule.

The term ‘strongly reducing agent’ in accordance with the present invention should be understood to mean a substance having a reduction potential of greater than +0.3V. This terminology is known to someone skilled in the art.

In preferred embodiments the moderately strong acid is hypophosphorous acid.

The moderately strong acid could also include oxalic acid or sulphurous acid however; it is an essential feature of the preferred acid that it is a combination of a strong acid of which the conjugate base is a weak nucleophile and which has very reducing properties. Oxalic acid has the same pKa and the same reduction potential as hypophosphorous acid, but its conjugate base provides a slightly stronger nucleophile.

Hypophosphorous acid (H₃PO₂) is cheap and readily available, and its residues are environmentally benign, which makes it preferable to any of the traditional production methods.

The applicant has found that the present invention has particular application to the formation of vicinal diols from lanosterol intermediates such as hydroxyhalogenated lanosterol or epoxidized lanosterol derivatives.

The bulk of the discussion in this specification shall now be directed to this application. Examples of other applications are discussed later on in the specification.

According to another aspect of the present invention there is provided a method of producing lanosterol derivative vicinal diols,

the method characterised by the step of

a) reacting a lanosterol intermediate with a moderately strong acid in the presence of one or more reagents capable of supplying hydroxyl groups
   wherein the 24,25-position of the lanosterol intermediate is reacted to produce the diol derivatives.

The term ‘lanosterol’ in relation to the present invention is defined as lanosta-8,24-diene-3β-ol, and is also known trivially as kryptosterol. Its molecular formula is C₃₀H₅₀O, and its molecular weight is 426.70.

In preferred embodiments the vicinal diol formed from the intermediate lanosterol derivative forms at the 24,25-position on the lanosterol derivative.

The term ‘24,25’ is the term used to describe the carbons 24 (C-24) and 25 in a molecule, in this case a steroid, and the nomenclature for counting carbon atoms in a steroid molecule is known to someone skilled in the art.

The term ‘lanosterol intermediate’ in accordance with the present invention will, in preferred embodiments, be either a diastereomeric mix of hydroxyhalogenated lanosterol derivatives or lanosterol derivative epoxides or the individual 24(R) or 24(S) epimers thereof.

The term ‘hydroxyhalogenated’ in accordance with the present invention should be understood to mean the presence of both a hydroxyl group and a halogen atom on vicinal carbons in a compound. This can include any member of the halogen series, those being fluorine, chlorine, bromine, or iodine.

In preferred embodiments, hydroxyhalogenation of lanosterol produces ‘24Halo-24 hydroxy-lanosterol derivatives, where the term ‘Halo’ is a general term to describe the inclusion of any member of the halogen series.

In preferred embodiments, the halogens of choice are iodine and bromine and chlorine.

The term ‘epoxide’ in accordance with the present invention should be understood to mean a compound that contains an oxirane three membered ring containing an oxygen and two carbons, and in this case involves the bridging of oxygen across two carbon atoms that are part of a chain.

In preferred embodiments, the opening of the lanosterol derivative epoxide bond is undertaken by reacting the epoxide with hypophosphorous acid in the presence of water and an organic reagent. As hypophosphorous acid is the moderately strong acid, as described earlier, it exhibits the required parameters of being strongly reducing while having a conjugate base that is a weak nucleophile.

In preferred embodiments the water-soluble organic reagent is 2-propanol, other organics reagents such as methanol and ethanol might be used but there is a risk of alkylation rather than hydroxylation with primary alcohols.

Lanosterol and its major impurity, dihydrolanosterol have physical and chemical properties that are very similar. This similarity is what makes them very difficult to separate. By synthesising certain intermediates of lanosterol, including vicinal diol derivatives, the difference in properties between dihydrolanosterol and the intermediates is maximised, making it possible to separate them by standard, well known methods. This provides a distinct advantage over current methods, as not only is the process of producing lanosterol an environmentally ‘green’ one (especially in comparison with mercury or osmium based reaction routes), but it also utilises standard separation techniques.

The separation of impurities from desired products can be undertaken by fractional crystallisation or flash column chromatography, but these are listed by way of example only and should not be seen to be limiting in any way.

The term ‘impurities’ in accordance with the present invention should be understood to mean any material contained within the reaction system that is not the desired end product, in this case anything that is not lanosterol or a mixture of lanosterol derivatives.

In particular, impurities can include dihydrolanosterol and derivatives thereof, but can also include agnosterol and dihydroagnosterol and derivatives thereof.

The application of hypophosphorous acid in the presence of water and an organic reagent to produce vicinal diols is new to steroid chemistry and has never before been available to scientists.

According to another aspect of the present invention there is provided a method of producing lanosterol characterised by the steps of
a) separating lanosterol derivative vicinal diols from impurities, and
b) converting the lanosterol derivative vicinal diols back to lanosterol.

In one embodiment, the term ‘converting’ in accordance with the present invention is the reacting of lanosterol derivative vicinal diols with N,N-dimethylformamide dimethylacetal in the presence of dichloromethane. The reaction converts the lanosterol derivative diols back to lanosterol acetate, which is then converted back to lanosterol by the use of refluxing with ethanolic potassium hydroxide.

The advantage of producing lanosterol derivative diols is that they can be separated from impurities, as discussed previously. Lanosterol occurs in a natural mixture with dihydrolanosterol. This occurrence has the disadvantage of providing researchers and industry alike with an impure starting material thereby reducing yields.

According to another aspect of the present invention there is provided a method of producing lanosterol characterised by the steps of
a) converting a lanosterol derivative epoxide to a vicinal diol, and
b) converting the vicinal diol to lanosterol.

The separation of lanosterol from dihydrolanosterol has not been undertaken in a ‘green’ and easily achieved manner before. The advantages provided by the above-described method are that high purity lanosterol (free from the dihydrolanosterol and other steroid impurities) is available as a starting material. The method described is simple and provides high purity yields of up to virtually 100% purity.

Literature yields include:
75% [Rodewald, W. J., Jagodzinski, J. J. A new method of isolating lanosterol from isocholesterol, Polish J. Cites, 1978, 52, 2473-2477], see discussion of environmental hazards of their method earlier.
   31% (for the free sterol 1 where R═OH) [Johnston, J. D., Gautschi, P., Bloch, K, Isolation of lanosterol from “isocholesterol”, J. Biol. Chem., 1957, 224, 185-190] 33% [Lewis, D. A., McGhie, J. F., Isolation and reactions of lanost-8:24-dien-3□-ol, Chem. Ind., 1956, 550-551.]
   36% (for the free sterol 1 where R═OH) [Maienthal, M., Franklin, P. J., Preparation of Lanosterol from bromo-lanosterol, J. Org. Chem. 1955, 20, 1627-1630.]

In some embodiments of the present invention, the diols formed are not converted back to lanosterol. As discussed earlier in the specification, vicinal diols are useful intermediates for the synthesis of biologically active compounds.

The diols do not need to be converted back to lanosterol in order to produce a commercially viable product. Instead, the diols can be converted directly to the desired end product. This may be done immediately follow diol production, or at a later stage.

According to another aspect of the present invention there is provided a method of producing vicinal diol derivatives of cyclohexane,

the method characterised by the step of

a) reacting an intermediate cyclohexane solution with a moderately strong acid in the presence a reagent capable of supplying hydroxyl groups
   wherein the moderately strong acid attacks the substituted position of the intermediate cyclohexane solution to produce the diol derivatives.

The term ‘cyclohexane’ in relation to the present invention is defined as a cyclic alkane containing 6 carbons. Its molecular formula is C₆H₁₂, and its molecular weight is 84.16.

In preferred embodiments, the vicinal diol formed from the intermediate cyclohexane solution forms at the vicinally substituted position on the cyclohexane derivative.

The term ‘intermediate cyclohexane solution’ in accordance with the present invention will, in preferred embodiments, be either a diastereomeric mix of hydroxyhalogenated cyclohexane derivatives or a diastereomeric mix of 1,2-epoxy-cyclohexanes.

In preferred embodiments, the hydroxyhalogenated cyclohexane derivatives include trans-2-bromocyclohexanol, trans-2-iodocyclohexanol and trans-2-chlorohexanol.

Advantages of producing vicinal diols of cyclohexane in accordance with the present invention is mild reaction conditions, low toxicity, inexpensive chemical reagents and excellent yields.

### BRIEF DESCRIPTION OF DRAWINGS

Further aspects of the present invention will become apparent from the following description which is given by way of example only and with reference to the accompanying drawings in which:

FIG. 1 is an illustration of a preferred embodiment of the present invention showing a scheme for the conversion of epoxide intermediates of lanosterol to diols.

FIG. 2 is an illustration of a preferred embodiment of the present invention showing a scheme for the conversion of hydroxyhalogenated intermediates of lanosterol to lanosterol diols.

FIG. 3 is an illustration of a preferred embodiment of the present invention showing a scheme for the conversion of 1,2-hydroxyhalogenated or 1,2-epoxyderivatives of cyclohexane to 1,2-cyclohexandiols.

### BEST MODES FOR CARRYING OUT THE INVENTION

Discussed below is one example of how the present invention has been used to produce lanosterol.

¹H and ¹³C NMR spectra were recorded in chloroform-dₗ using a Bruker ADV DRX 400 MHz Spectrometer. Mass spectra (MS) were determined on a HP5970B spectrometer at ionising voltage of 70 eV interfaced with an ultra HP5890 gas chromatograph fitted with an HP-1 column (25 m×0.22 mm). Column chromatography was performed on Merck silica gel (70-230 mesh). Thin layer chromatography was carried out on Merck precoated silica gel 60 F₂₅₄ plates (0.25 mm thick). Gas chromatography was performed on a HP5890 Gas chromatograph fitted with an ultra HP Ultra 2 column (25 m×0.32 mm). All melting points were obtained on a micro-melting point determination apparatus and were uncorrected. Lanosterol (62% purity) was purchased from Sigma and used as such. All commercial reagents were used as such, without purification.

All numbers that appear in bold relate to the figures. A number 1 is molecule number one, and so forth.

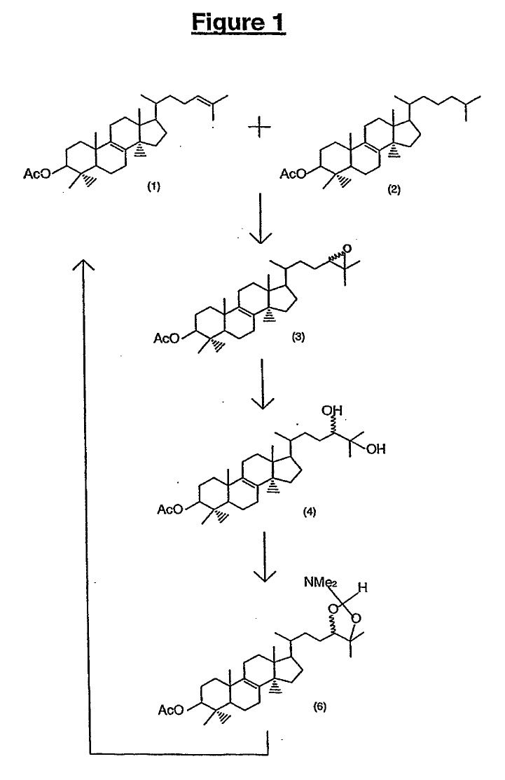

A) FIG. 1 (The Epoxide Route—Late Separation)

To a solution of the impure lanosterol acetates (1 and 2) (20 g—Lanosterol acetate 12.3 g, dihydrolanosterol acetate 7.7 g) in dichloromethane (600 mL) a mixture of m-chloroperbenzoic acid (70% pure) (5.5 g) and sodium hydrogen carbonate (2.2 g, 0.05 mol) was added in the following manner:

Half was added at room temperature over 3 hr and the remainder at 0° C. (ice bath), also over 3 hr. The mixture was stirred vigorously (1 hr) and left in a refrigerator overnight. The reaction mixture was diluted with 2-propanol (500 mL), hypophosphorous acid (20 mL) and water (200 mL). Dichloromethane was removed at atmospheric pressure, an additional amount of hypophosphorous acid (10 mL) added and the mixture heated to reflux (2.5 hr).

The reaction mixture was poured into water, filtered and washed with water. Purification by short column chromatography (dichloromethane) yielded dihydrolanosterol acetate (2) (7.3 g, 95%), mp 120-121° C. Further elution with ethyl acetate afforded the diol (4) as colourless solid (10.7 g, 82% calculated from 100% of (1), mp 173-176° C. (Raab, K. H. et. al., Biochim. Biophys. Acta [1968] 152, 742-748 gives 166-169° C. and Sato, Y & Sonoda, Y., Chem. Pharm. Bull., [1981], 29 356-365 gives 183-185° C.). Anal. Calcd for C₃₂H₅₄O₄: C, 76.44; H, 10.83. Found C, 76.52; H, 10.84.

B) FIG. 1 (The Epoxide Route—Early Separation)

To a solution of the impure lanosterol acetate (1 and 2) (20 g—Lanosterol acetate 12.3 g, dihydrolanosterol acetate 7.7 g) in dichloromethane (600 mL) a mixture of m-chloroperbenzoic acid (70% pure) (5.5 g) and sodium hydrogen carbonate (4.2 g, 0.05 mol) was added in the following manner: half was added at room temperature over 3 hours and the remainder at 0° C. (ice bath), also over 3 hours. The mixture was stirred vigorously (1 hour) and left in a refrigerator overnight. The mixture was washed with water, dried (MgSO₄) and after evaporation of the solvent the crude residue was purified by flash column chromatography (50% dichloromethane in light petroleum) giving dihydrolanosterol acetate (2) (7.4 g 96%) as a colourless solid. Further elution with dichloromethane afforded a white solid (10.8 g, 85% calcd. From 100% of (1) which was crystallised from acetone to give colourless needles of 24(R,S)-3β-acetoxy-24,25-epoxy-5α-lanost-8-ene, (3), mp 172-180° C. Anal. Calcd for C₃₂H₅₂O₃: C, 79.35; H, 10.73. Found C, 79.22; H, 10.66. MS m/z 484(M⁺), 469, 451, 409 (M⁺-75, base peak), 391.

To a solution of the epoxy acetates (3) (10 g, 0.02 mol) in 2-propanol (250 mL) was added water (100 mL), hypophosphorous acid [18 mL (50% in water)] and the mixture was heated to reflux (3 hr), then diluted with water, filtered, washed and, after crystallisation from aqueous acetone, afforded 24(R,S)-3β-acetoxy-24,25-dihydroxy-5α-lanost-8-ene (4) (9.7 g, 94% based on 3) as colourless needles.

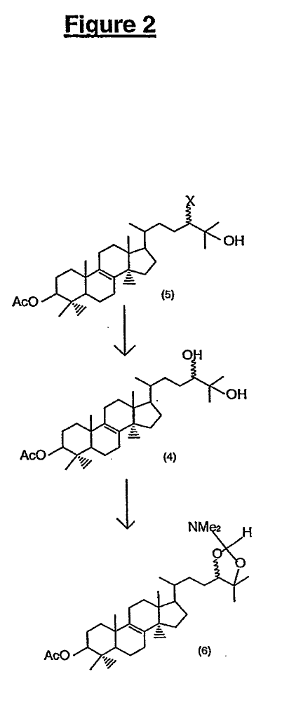

C) FIG. 2 (The Halo [Bromo] Hydroxy Route)

A reaction mixture containing the Halo [Bromo] hydroxy lanosterol (5 where X=Br) was stirred at room temperature (15 min), NaHCO₃ (3 g) was added and the reaction mixture concentrated under vacuum. The residue was dissolved in 2-propanol (300 mL), water (100 mL), hypophosphorous acid [7.2 mL (50% in water)] and an additional 4.5 g of NaHCO₃ was added. The reaction mixture was refluxed (4 hr), then diluted with water, filtered and washed until neutral. Separation by flash column chromatography (dichloromethane) yielded dihydrolanosterol, with minor by-products (3.5 g, 92%). Further elution with ethyl acetate afforded white solid 24(R,S)-3β-acetoxy-24,25-dihydroxy-5α-lanost-8-ene (4) (5.8 g, 88% calcd from 100% of acetylated lanosterol).

D) FIG. 2 (The Halo [Iodo] Hydroxy Route)

A reaction mixture containing the Halo [Iodo] hydroxy lanosterol (5 where X=I) was stirred at room temperature (10 min), NaHCO₃ (3 g) was added and the reaction mixture concentrated under vacuum. The residue was dissolved in 2-propanol (300 mL), water (100 mL), hypophosphorous acid (7.2 mL (50% in water)) and added additional NaHCO₃ (4.5 g). The reaction mixture was refluxed (3 hr), then diluted with water, filtered and washed until neutral. Separation by flash column chromatography (dichloromethane) yielded dihydrolanosterol (2), with minor by-product (3.5 g, 91%). Further elution with ethyl acetate afforded white solid 24(R,S)-3β-acetoxy-24,25-dihydroxy-5α-lanost-8-ene (4) (5.7 g, 87% calcd from 100% of acetylated lanosterol).

E) Conversion of Vicinal Diols (4) into Lanosterol Acetate

To a solution of 24(R,S)-3β-acetoxy-24,25-dihydroxy-5α-lanost-8-ene, (4), (10 g) in dichloromethane (150 mL) was added N,N-dimethylformamide dimethylacetal (8.5 mL) and the mixture was refluxed (2.5 hr). The reaction mixture was cooled, acetic anhydride (20 mL) was added and dichloromethane was distilled under reduced pressure.

An additional amount of acetic anhydride (100 mL) was added and the mixture containing acetal (6) was refluxed at 130° C. (3.5 hr), then cooled, poured into ice-water and filtered and washed until neutral to yield light brown power. The crude product after quick chromatography through silica gel afforded lanosterol acetate, (8.3 g, 95%) as white powder, imp. 129.5-131.5° C. [α]_{D}=58.6 (C 1.16, chloroform). Merck Index m.p. 131.5-133° C. [α]_{D}=+62.9 (C 1.12, chloroform). ¹H and ¹³C NMR data corresponded to literature.

F) Conversion of Lanosterol Acetate (1) to 5α-lanosta-8,24-diene,3β-ol

The lanosterol acetate (1) (5 g, 98% purity by gas chromatography) was hydrolysed by refluxing with 10% ethanolic potassium hydroxide (150 mL) for 2.5 hr. The resulting mixture was poured into ice water and after standing 6-7 hours was collected by filtration, dried and recrystallised from acetone to yield 5 α-lanosta-8,24-diene-3β-ol (4.2 g, 94%) m.p. 139-140° C. Merck Index mp 138-140° C.

GC-Analysis for purity of lanosterol acetates obtaining by different routes gave: before (and after in parentheses) fractional crystallisation, and yield of acetylated lanosterol and [lanosterol] is shown in Table 1.

TABLE 1
Purity of Ianosterol acetate before and (after fractional crystallisation),
and yields of acetylated lanosterol and Ianosterol using the different
reaction routes.

| | | Yield of acetylated |
|---|---|---|
| | | Lanosterol 3-OAc, |
| Route | Purity | [Ianosterol 3-OH) |
| Epoxide route, late separation | 95% (98%), | 78% [73%] |
| Epoxide route, early separation | 97% (98.5%), | 76% [71%] |
| Bromohydroxy route | 97.5 (99.5%), | 84% [79%] |
| Iodohydroxy route | 97.7% (˜100%) | 76% [71%] |

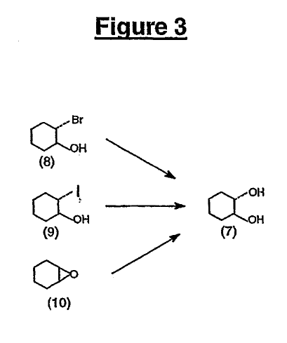

G) Preparation of 1,2-cyclohexanediol (7) from trans-2-bromocyclohexanol (8) (FIG. 3)

To a solution of trans-2-bromocyclohexanol (8) was added 2-propanol (20 mL), hypophosphorous acid (6.5 ml; 50% in water) and the pH of the solution adjusted to 6.5 using a saturated solution of NaHCO₃. The mixture was stirred at reflux (80° C.) for 4 h, cooled, diluted with ice water, filtered and washed with water. Water was removed from the filtrate under reduced pressure and the dry residue was dissolved in dichloromethane and purified by short column chromatography (elution with dichloromethane then with diethyl ether or ethyl acetate) to afford the diol (7) as a colourless solid (1 g, 93%), m.p. 102-104° C. (lit. 101-104° C.), [α]_{D}=0 (C 1.6, water). ¹H and ¹³C NMR data are presented in Table 2.

H) Preparation of trans-1,2-cyclohexanediol (7) from trans-2-iodocyclohexanol (9) (FIG. 3)

trans-2-iodocyclohexanol (9) and corresponding trans-1.2-cyclohexanediol (7) were synthesised according to the above procedures gave yields of 99% and 85% by GC respectively.

I) Preparation of 1,2-cyclohexanediol (7) from 1,2-epoxycyclohexane (10) (FIG. 3)

To a stirred solution of cyclohexane [1 mL (0.807 g, 0.0098 mol)] in acetone (5 mL) and water (15 mL) was added a mixture of m-chloroperbenzoic acid 2.9 g (70% pure) and NaHCO₃ (1 g, 0.0119 mol) at 0° C. over 10 min and stirring continued for 1 hr at room temperature. 2-Propanol (10 mL), hypophosphorous acid (6.5 ml; 50% in water) were added and after distillation of the acetone the reaction mixture was refluxed (80° C.) for 1 hr, cooled, neutralized with a saturated solution of Na₂CO₃ and concentrated under reduced pressure. The dry residue was dissolved in dichloromethane and purified by short column flash chromatography (elution with dichloromethane then with ether or ethyl acetate) to afford the diol (7) as a colourless solid (1.08 g, 95%).

TABLE 2
¹H and ¹³C NMR data for 1,2-cyclohexanediol (7) (from epoxide,
hydroxy bromide and iodide)

| | Trans-1,2-cyclohexanediol (from epoxide, | |
|---|---|---|
| No | Hydroxy bromidce and iodide) | |
| Carbon | ¹³C | ¹H |
| 1 | 75.64 | 3.284 (m) |
| 2 | | J_{1.6/2.3} = 12.5 Hz |
| 3 | 32.92 | 1.906 eq |
| | | 1.204 ax |
| 4 | 224.38 | 1.64 eq |
| 5 | | 1.204 ax |
| 6 | 32.92 | 1.906 eq |
| | | 1.204 ax |

Aspects of the present invention have been described by way of example only and it should be appreciated that modifications and additions may be made thereto without departing from the scope thereof as defined in the appended claims.

1. A method of producing vicinal diols from a compound, the method characterised by the step of:
a) reacting the compound with a moderately strong acid in the presence of one or more reagents capable of supplying hydroxyl groups,
wherein the moderately strong acid is a strongly reducing agent, but has a conjugate base that is a weak nucleophile, and wherein the compound is cohalogenated or contains a carbon-carbon multiple bond.

2. A method as claimed in claim 1, wherein the compound is lanosterol.

3. A method as claimed in claim 1, wherein the compound is a lanosterol intermediate.

4. A method as claimed in claim 1 wherein the compound is cyclohexene or a derivative thereof.

5. A method as claimed in claim 1, wherein the compound is hydroxyhalogenated.

6. A method as claimed in claim 1 wherein the compound is an individual epimer.

7. A method as claimed in claim 1 wherein the moderately strong acid has:
a) a pKa of less than or equal to 2.0, and
b) a reduction potential of greater than or equal to +0.3V.

8. A method as claimed in claim 1 wherein the moderately strong acid is hypophosphorous acid.

9. A method as claimed in claim 1 wherein the moderately strong acid is oxalic acid.

10. A method as claimed in claim 1 wherein the moderately strong acid is sulphurous acid.

11. A method as claimed in claim 1 wherein one of the reagents is water.

12. A method as claimed in claim 1 wherein one of the reagents is organic.

13. A method as claimed in claim 12 wherein the reagent is water soluble.

14. A method as claimed in claim 12 wherein the reagent is 2-propanol.

15. A method of producing vicinal diols as claimed in claim 1 where the compound is lanosterol or a lanosterol intermediate, the method characterised by the step of
a) reacting the compound with a moderately strong acid in the presence of one or more reagents capable of supplying hydroxyl groups
wherein the 24,25-position of the compound is reacted to produce the vicinal diols.

16. A method as claimed in claim 15 wherein the compound is a diastereomeric mix of hydroxyhalogenated lanosterol derivatives.

17. A method as claimed in claim 15 wherein the compound is a diastereomeric mix of lanosterol derivatives.

18. A method as claimed in claim 15 wherein the compound is the individual 24(R) or 24(S) epimers thereof.

19. A method of removing impurities from lanosterol characterised by the steps of
a) forming lanosterol intermediate vicinal diols using the method of claim 1,
b) separating the lanosterol intermediate vicinal diols from the impurities, and
c) converting the lanosterol intermediate vicinal diols to lanosterol.

20. A method of producing lanosterol, characterised by the steps of
a) separating lanosterol derivative vicinal diols from impurities as claimed in claim 19, and
b) converting the lanosterol derivative vicinal diols back to lanosterol.

21. A method of reacting lanosterol derivative vicinal diols to produce lanosterol wherein the vicinal diols are produced by the method as claimed in claim 1, the method characterised by the steps of
a) reacting the lanosterol derivative vicinal diols with N,N-dimethylformamide dimethylacetal in the presence of dichloromethane to produce lanosterol acetate, and
b) converting the lanosterol acetate back to lanosterol by refluxing with ethanolic potassium hydroxide.

22. A method of producing lanosterol acetate characterised by the step of
a) reacting lanosterol derivative vicinal diols with N,N-dimethylformamide dimethylacetal in the presence of dichloromethane to produce lanosterol acetate.

23-27. (Canceled)
